(19) United States
(12) Patent Application Publication
(10) Pub. No.: US 2005/0038289 A1
(43) Pub. Date: Feb. 17, 2005

(54) Chemical reactions in compressed carbon dioxide

(76) Inventors: Richard S Gordon, Cambridge (GB); Tessa Early, Cambridge (GB); Andrew Holmes, Cambridge (GB)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/484,457

(22) PCT Filed: Jul. 23, 2002
(86) PCT No.: PCT/GB02/03361

(30) Foreign Application Priority Data
Jul. 23, 2001 (GB) 0117846.6

(51) Int. Cl. C07C 51/10; C07C 51/14

(52) U.S. Cl. 562/517

(57) ABSTRACT

A palladium-catalysed carbon-carbon bond forming reaction in compressed carbon dioxide is provided wherein at least one of the reagents used in said reaction is bounded to a solid polymer support. In a second aspect, a palladium-catalysed carbon-carbon bond forming reaction in compressed carbon dioxide is provided wherein said reaction is performed in the presence of a tetra-alkylammonium acetate. In a third aspect, a palladium-catalysed carbon-carbon bond forming reaction in compressed carbon dioxide is provided wherein said palladium catalyst does not have any fluorinated phosphine ligands but does have at least one phosphine ligand that has at least one substituent that is selected from tert-alkyl groups, cycloalkyl groups and optionally substituted phenyl groups or 1′-diphenylphosphino-biphenyl. In a fourth aspect, there is provided a palladium-catalysed Suzuki or Heck reaction in compressed carbon dioxide wherein both of the substrates being combined in said reactions are boronic acids.

### FIELD OF INVENTION

The present invention relates to palladium-catalysed cartoon-carbon bond forming reactions in compressed carbon dioxide.

### BACKGROUND TO THE INVENTION

Supercritical carbon dioxide has been used for polymer synthesis and polymer processing. This has been extensively reviewed in the past and the state of the art is summarised in an article by Cooper [A. I. Cooper, J. Mater. Chem., 2000, 10, 207]. Compressed carbon dioxide is also used as a solvent for the preparation of organic molecules and this has been summarised in a special issue of Chemical Reviews. [see Special Issue: Chem. Rev. 1999, 99, #2]. Unlike conventional liquid solvents, carbon dioxide is highly compressible and the density (and therefore solvent properties) can be tuned over a wide range by varying the pressure [see M. McHugh et al. “Supercritical Fluid Extraction” Boston, Butterworth-Heinemann, 1994]. Compressed carbon dioxide is an attractive alternative to conventional solvents because it is inexpensive, non-toxic and non-flammable. Compressed carbon dioxide reverts to the gaseous state upon decompression, simplifying solvent separation from solute(s) and reaction products.

Metal-catalysed processes are extremely common in the synthesis of small organic molecules for the pharmaceutical industry as well as for agrochemicals, flavours, fragrances and specialist consumer products. They are assuming growing importance in the synthesis of macromolecules, particularly conjugated polymers (see for example Bernius, M. T.; Inbasekaran, M.; Brien, J.; Wu, W. Adv. Mater., 2000, 12, 1737).

Metal-catalysed homogeneous reactions in supercritical carbon dioxide have been reported and the state of the art is summarised in a special edition of Chemical Reviews, 1999, 99(#2) and in a monograph “Chemical synthesis using supercritical fluids” P. G. Jessop and W. Leitner, Wiley-VCH, Weinheim, 1999. A comprehensive review of organic synthesis in supercritical carbon dioxide bas been written by Oakes, R. S.; Clifford, A. A.; Rayner, C. M. J. Chem. Soc., Perkin 1, 2001, 917. WO-A-98/32533 discloses the use of phosphorus ligands carrying perfluoroalkyl chains to solubilise rhodium phosphine complexes in hydroformylation and hydrogenation reactions. WO-A-99/38820 discloses the use of ligand-metal complexes in which the complex comprises a perfluorinated group for the transformation of organic molecules. In some of the reactions, the substrate was anchored to a solid polymer support. Palladium-catalysed cross coupling reactions in supercritical carbon dioxide have been disclosed (M. A. Carroll. M. A.; Holmes A. B. Chem. Commun., 1998, 1395; Morita, D. K; Pesiri, D. R; David, S. A.; Glaze, W. H.; Tumas, W.; Chem. Commun., 1998, 1397; Shezad, N., Oakes, R. S., Clifford, A. A. and Rayner, C. M., Tetrahedron Lett., 1999, 40, 2221). The first mentioned paper reported Heck, Suzuki (Suzuki, A in Metal-catalysed Cross-coupling reactions, eds. Diederich, F. and Stang, P. J., Wiley-VCH, Weinheim, 1997.) and Sonogashira reactions. The second reported, in addition, Stille reactions while the third reported the use of palladium(II) trifluoroacetate as the catalyst source. These examples teach that perfluorinated alkyl ligands (or trifluoroacetate) are needed, presumably for enhancement of solubility of the complex in supercritical carbon dioxide (sc CO₂). Non-fluorinated triarylphosphines have been shown to lead to lower conversions in the Heck reaction (the palladium-mediated addition of an aryl or vinyl halide to an alkene with regeneration of the double bond in the original alkene partner; see Palladium reagents in organic synthesis”, R. F. Heck, Academic Press, Orlando, 1985; Heck, R. F., Org. React., 1982, 27, 345; Beletskaya, I.; Cheprakov, Chem. Rev., 2000, 100, 309). Exceptionally, ring closing olefin metathesis (R. H. Grubbs and S. Chang, Tetrahedron, 1998, 54, 4413-4450) with an insoluble diphenylalkylidene ruthenium catalyst has been realised. (Fürstner, A. et al. Angew. Chem. Int. Ed., 1997, 36, 2646).

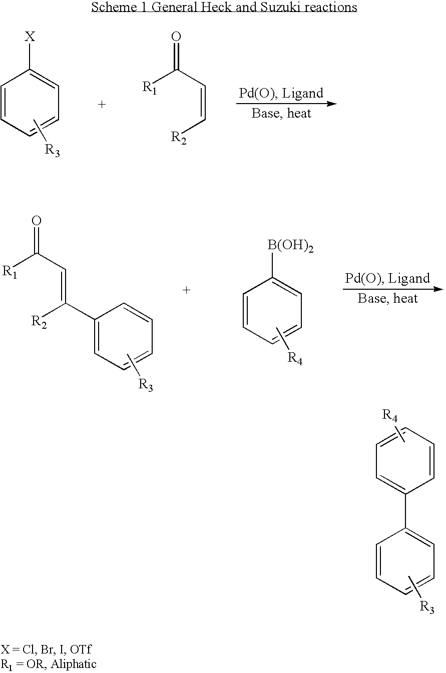

### SUMMARY OF THE INVENTION

An important aspect of all the above palladium-mediated carbon-carbon bond forming reactions was the need for a solubilising fluorine-containing phosphine ligand or a trifluoroacetate counterion to for a homogeneous solution of the palladium complex in compressed carbon dioxide. WO-A-99/38820 discloses the use of perfluorinated ligand-palladium complexes in which some of the reactions were performed using a substrate which was anchored to a solid polymer support. It has never been previously disclosed or suggested however, that it might be possible to perform palladium-mediated carbon-carbon bond forming reactions in compressed carbon dioxide using reagents that are anchored to a solid polymer support. We have now made the surprising discovery that it is possible to perform such palladium-mediated reactions in compressed carbon dioxide using reagents immobilised on commercially available solid supports. This gives many advantages, including increased ease of processing making it attractive as a potential means of performing these reactions on an industrial scale and the discovery that using such reagents it is possible to obtain excellent yields without the need for a fluorinated phosphine ligand.

Thus, in a first aspect of the present invention there is provided a palladium-catalysed carbon-carbon bond forming reaction in compressed carbon dioxide as a solvent wherein at least one of the reagents used in said reaction is bound to a solid polymer support.

By compressed carbon dioxide, we mean carbon dioxide which has been compressed under pressure to produce liquid carbon dioxide or supercritical carbon dioxide. A supercritical fluid may be defined as a substance for which both the temperature and pressure are above the critical values for the substance and which has a density close to or higher than the critical density. Carbon dioxide is an ideal solvent because of its mild critical temperature (31.1° C.) and its relatively low critical pressure (73.8 bar). Another advantage of carbon dioxide is that it is a gas under atmospheric conditions so that the end-reaction mixture is solvent-free.

By reagents we mean agents that are used to enable the palladium-catalysed reaction to be performed but which do not include the actual substrates that are being coupled in the carbon-carbon bond forming reaction. Examples of the polymer-bound reagents include polymer-supported bases and polymer-supported solubilising ligands. By solubilising ligands we mean reagents that can interact with the source of palladium in the reaction and, as a result, increase the solubility of the palladium in the compressed carbon dioxide.

Typical examples of the polymer-supported bases include polymer-supported amine bases such as polymers hang supported monoalkylaminoalkyl groups and polymers having supported dialkylaminoalkyl groups wherein each alkyl group is the same or different and preferably has from 1 to 6 carbon atoms. Typical examples of the supporting polymer include polystyrenes and macroreticular resins (e.g. Amberlyst®). Preferred examples of the polymer-supported bases include dialkylaminoalkylpolystyrene and dialkylamino-macroreticular resin, of which diethylaminomethylpolystyrene, diethylaminomethyl-Amberlyst resin and a disopropylmethylaminopolystyrene are more preferred and diethylaminomethylpolystyrene is most preferred. Further examples of suitable polymer-supported bases are found in the review by S. V. Ley et all J.C.S. Perkin Trans. I, 2000, 3815. Using polymer-supported bases such as these we have found that it is even possible to perform palladium-catalysed carbon-carbon bond forming reactions without the need for the addition of a phosphine ligand to the palladium source.

Typical examples of polymer-supported solubilising ligands are polymer-supported phosphine ligands. Typical examples of the supporting polymer include polystyrenes and macroreticular resins (e.g. Amberlyst®). The phosphine ligands include ones that have at least one fluoro-substituted aliphatic or aromatic substituent such as phosphines that have at least one C₁-C₂₀ perfluoroalkyl substituent such as a 1H,1H,2H,2H-perfluorooctyl group but also include ones that do not have at least one fluorinated substituent but instead have substituents such as alkyl groups (e.g. alkyl groups having from 1 to 6 carbon atoms, especially t-alkyl), cycloalkyl groups such as those having from 3 to 8 carbon atoms and aryl groups such as phenyl groups which can be substituted with at least one alkyl group having from 1 to 6 carbon atoms. Preferred examples include polymers having supported diarylphosphinoalkyl groups, polymers having supported dialkylphospinoalkyl groups and polymers having supported dicycloalkylphospinoalkyl groups wherein the alkyl, cycloalkyl and aryl groups are as defined above. More preferred examples include polystyrenes and macroreticular resins having supported diphenylphosphinoalkyl groups, and the most preferred example is diphenylphosphinomethylpolystyrene. Polymer-supported phosphines are well-known in the art and are discussed, for example, in Trost et al, J. Am. Chem. Soc., 1978, 100, 7779, and Jang, Tetrahedron Lett., 1997, 38, 1793, and can be obtained commercially (e.g. from Nova Biochem).

Not only is there significant potential for catalyst recyclability (as the palladium is anticipated to remain on the resin) but these reactions could potentially be monitored by on line procedures.

During our work, we have found that polymer-supported reagents such as polymer supported bases and polymer-supported solubilising phosphines allow completely heterogeneous Heck and Suzuki coupling reactions to be carried out in compressed carbon dioxide. Based on this observation, all polymer-supported reagents should show significant enhancements in rates, reactivity and yields when used in these palladium-mediated carbon-carbon bond forming reactions. Examples of reactions which can all show improved yields through the use of polymer-supported reagents include Heck reactions (e.g. see “Palladium reagents in organic synthesis”, R. F. Heck, Academic Press, Orlando, 1985; Heck, R. F., Org. React., 1982, 27, 345; and Beletskaya, I.; Cheprakov, A. Chem. Rev., 2000, 100, 309), Suzuki reactions (e.g. see Migaura et al, Syn. Commun, 1981, 11, 513), Sonogashira reactions (e.g. see Sonogashira et al, Tetrahedron Lett, 1983, 4467) and Stille reactions and related reactions.

As one example, we found that for the Heck reaction the addition of aryl iodide and bromide substrates to acrylates such as butyl acrylate was surprisingly effective using the combination of a palladium (0) source such as palladium (II) acetate and a polymer-supported base. Suitable polymer-supported bases are of the type described and exemplified above, of which we found that ones selected from diethylaminomethylpolystyrene, a diethylaminomethyl-Amberlyst resin and a disopropylmethylaminopolystyrene are preferred and diethylaminomethylpolystyrene is most preferred. Using these bases, we found that no solubilising phosphine ligand is required at all. For carbon-carbon bond forming reactions such as the Heck reaction to have useful application in the pharmaceutical industry, it is essential that the phosphine content and the fluorine-substituted solubilising groups should be kept to a minimum. Clearly, the ability to perform these reactions with no phosphine ligands or fluorine-substituted solubilising groups in some circumstances makes the process of the present invention highly promising. Furthermore, an inherent feature of this invention is that the heterogeneous reaction with palladium catalyst leaves the catalyst embedded in the polystyrene matrix. Experiments, using the recycled resin, are encouraging. This invention therefore shows the potential for a continuous flow process using recycled palladium/resin.

Again using the Heck reaction as an example, although the combination of simple Pd (II) salts such as palladium acetate and aryl halides had been previously used in the phase-transfer catalysis version of the Heck reaction (Jeffery, T. Tetrahedron 1996, 30, 10113), we have surprisingly found that the Heck addition of aryl iodides or bromides to an acrylate such as butyl acrylate can be realised in the presence of a polymer-supported solubilising phosphine ligand. Suitable polymer-supported phosphine ligands are of the type described and exemplified above, of which diphenylphosphinomethylpolystyrene is particularly preferred. The Heck addition using a palladium source such as palladium (E) acetate in the presence of a polymer-supported phospine ligand is best performed in the presence of a base or another promoting additive, preferred examples of such bases and additives including: diisopropylethylamine, cesium carbonate, polymer-supported bases of the type described above such as diethylaminomethylpolystyrene, sodium acetate, sodium trifluoroacetate, triethylamine, tri-n-butylamine, perfluorinated trihexylamine, polystyrenemethylammonium carbonate, tetramethylethylamine diamine (TMEDA), tetramethylhexanediamine, and tetraalkylaminonium acetates such as tetrabutylammonium acetate. Of these, we prefer diisopropylethylamine and tetaalkylammonium acetates. Most preferably the additive is tetrabutylammonium acetate.

The combination of tetrabutylammonium chloride in the palladium-mediated Stille reaction with various phosphine ligands in super critical CO₂ has been reported (Osswald, T.; Schneider, S.; Wang, S.; Bannwarth, W. Tetrahedron Lett, 2001, 42, 2965). This teaches the use of this additive, but surprisingly we have now found that tetra-alkylammonium acetates give significantly superior yields in palladium-catalysed carbon-carbon bond forming reactions.

Thus, in a second aspect of the present invention, there is provided a palladium-catalysed carbon-carbon bond forming reaction in compressed carbon dioxide as a solvent wherein said reaction is performed in the presence of a tetra-alkylammonium acetate. Each alkyl group can be the same or different and typically has from 1 to 6 carbon atoms, of which alkyl groups having from 1 to 4 carbon atoms are more preferred. Preferred are tetraethylammonium acetate and tetra(n-butyl)ammonium acetate, of which tetra(n-butyl)ammonium acetate is particularly preferred.

We have surprisingly found that the yields in palladium-catalysed carbon-carbon bond forming reactions are significantly superior when a tetra-alkylammonium acetate is added to the reaction mixture in place of the known tetra-alkylammonium chlorides. While not wishing to be bound by theory, we believe that the role of the tetra-alkylammonium acetates is probably related to interfacial catalysis.

Typically, the palladium-mediated carbon-carbon bond forming reaction is conducted using a solubilising ligand such as a phosphine ligand. Examples include include ones that have at least one fluoro-substituted aliphatic or aromatic substituent such as phosphines that have at least one C₁-C₂₀perfluoroalkyl substituent such as a 1H,1H,2,2H-perfluorooctyl group but also include ones that do not have at least one fluorinated substituent but instead have substituents such as alkyl groups (e.g. alkyl groups having from 1 to 6 carbon atoms, especially t-alkyl), cycloalkyl groups such as those having from 3 to 8 carbon atoms and aryl groups such as phenyl groups which can be substituted with at least one alkyl group having from 1 to 6 carbon atoms. Preferred examples include tri(t-butyl)phosphine, tri(cyclohexyl)phosphine, tri(o-tolyl)phosphine and 1′-diphenylphosphinobiphenyl. Particularly preferred, however, are polymer-supported solubilising ligands such as the polymer-supported phosphines described and exemplified above. Of these, we particularly prefer polystyrenes and macroreticular res having supported diphenylphosphinoalkyl groups, and the most preferred example is diphenylphospinomethylpolystyrene.

The use of tetra-alkylammonium acetates at elevated temperatures leads to a two-phase reaction medium involving the molten ammonium salt as one component. A further aspect of this invention is that the tetra-alkyl ammonium salts (e.g tetraethyl) may be used as hydrates and the tetra-alkylammonium salts such as the acetates may be used as 1M aqueous solutions, providing a multiphase reaction medium for the actual carbon-carbon bond forming reactions.

A feature of the first aspect of the invention using polymer-supported reagents is the, ease of isolation of product. This follows from a simple washing of the reaction cell with compressed carbon dioxide which selectively removes the small molecule product from the solid phase reactants. In the case of the Suzuki reaction it has been demonstrated that the biaryl product can be simply isolated by washing and extraction in compressed carbon dioxide, followed by venting the pressure. This operation shows considerable promise for continuous flow manufacturing in small bore carbon dioxide reactors where the catalyst and an amine salt remain behind in the reactor vessel and the product is enacted by carbon dioxide. It is also expected that this process should be equally possible using the tetralkylammonium additive technique.

Thus, in a preferred embodiment of the present invention, there is provided a palladium-mediated carbon-carbon bond forming reaction according to the first or second aspects of the present invention defined and exemplified above wherein said reaction is conducted as a continuous flow reaction. Surprisingly the delivery of the reagents and compressed CO₂ solvent through a mixing nozzle into a reaction tube, previously charged with the catalyst, leads to an extremely rapid chemical reaction under conditions above the critical temperature and pressure. The products and unconverted starting materials emerge from the reactor through a filter. The rate of flow of the products may be controlled, for example, by the use of a back pressure regulator. This procedure can be applied to Suzuki reactions (e.g. an aryl halide coupled with a boronic acid), Heck reactions (e.g. an aryl halide coupled with an olefin), Sonogashira reactions (e.g. an aryl halide coupling with an alkyne) and Stille reactions (e.g. an aryl halide coupled with an organostannane). Most preferably the Suzuki reaction can be carried out under continuous flow conditions. Rapid formation of product is observed even when the reactants are subject to a single pass through the reactor, involving a short residence time in contact with the catalyst. A co-solvent may be employed to assist in the charging of the reactor with some reagents. Preferred co-solvents include methanol and toluene, but any selection of common solvents, including fluorinated solvents may be used.

As discussed above, it has previously been believed that phospine ligands having highly fluorinated substituents or trifluroacetate counterions were needed to enable palladium-catalysed carbon-carbon bond forming reactions to be performed in compressed carbon dioxide with acceptable yields. For example, previously the electron rich tri(2-furyl)-phosphine had been employed as a non-fluorinated ligand but with low yields (Morita, D. K.; Pesiri, D. R.; David, S. A; Glaze, W. H.; Tumas, W.; Chem. Commun., 1998, 1397). Surprisingly, we have now found that a range of non-fluorinated phosphine ligands in combination with a palladium (0) source such as palladium (II) acetate catalyse carbon-carbon bond forming reactions more efficiently than the combinations employing fluorinated phosphines.

Thus, in a further aspect of the present invention there is provided a palladium-catalysed carbon-carbon bond forming reaction in compressed carbon dioxide as a solvent wherein said palladium catalyst does not have any fluorinated phosphine ligands but does have at least one phosphine ligand that has at least one substituent that is selected from the group consisting of tert-alkyl groups having from 4 to 10 carbon atoms, cycloalkyl groups having from 3 to 8 carbon atoms and phenyl groups which can be substituted with at least one alkyl group haling from 1 to 6 carbon atoms or 1′-diphenylphosphinobiphenyl.

The solubility of triethylphosphine in compressed CO₂ and its use in certain hydroformylation reactions has previously been described by Cole-Hamilton (Bach, I.; Cole-Hamilton, D. J. Chem. Commun., 1998, 1463) but there has never previously been any suggestion that tert-alkylphospine solubilising groups might give superior results to perfluorinated alkylphospine solubilising groups in palladium-catalysed carbon-carbon bond forming reactions.

Preferred examples of tert-alkyl substituents for the phosphine ligands include tert-butyl groups, preferred examples of the cycloalkyl substituent are cyclohexyl groups and preferred examples of the optionally substituted phenyl groups are o-tolyl groups. Preferred phosphines include tri(t-butyl)phosphine, tri(cyclohexyl)phosphine, tri(o-tolyl)phosphine and 1′-diphenylphosphinobiphenyl groups. The most preferred phosphine ligand is tri(t-butyl)-phosphine.

The palladium source used as the catalyst is any suitable source of palladium (0). Preferred examples include palladium (II) acetate (by acetate we include both acetate per se and fluorinated acetates such trifluroacetate)

The Suzuki cross coupling of aryl bromides and iodides is effected in carbon dioxide in the presence of a palladium (0) source such palladium (II) acetate, non-fluorinated phosphines of the type defined and exemplified above [preferably selected from tri(t-butyl)phosphine, tri(cyclohexyl)phosphine, tri(6-tolyl)phosphine and 1′-diphenylphosphinobiphenyl] and a base or other reaction-promoting additive. Preferred examples of bases and other reaction-promoting additives include diisopropylethylamine, cesium carbonate, diethylaminomethylpolystyrene, sodium acetate, sodium trifluoroacetate, triethylamine, tri-n-butylamine, perfluorinated trihexylamine, polystyrenemethylammonium carbonate, tetramethylethylenediamine diamine (TMEDA), tetramethyl hexanediamine, and tetraalkylammonium acetates such as tetrabutylammonium acetate. Preferred bases are tetramethyl hexanediamine and cesium carbonate, the former being surprisingly soluble in compressed carbon dioxide. As alternatives to a base, the preferred reaction-promoting additives are tetra-alkylammonium acetates, particularly tetra(n-butyl)ammonium acetate.

An added feature of this invention is the use of biphasic conditions, for example the combination of water, methanol or isopropanol with compressed carbon dioxide. Water is preferred. For example, the combination phenyl boronic acid, bromobenzene, cesium carbonate, palladium (II) acetate, phosphine, and water (10 vol %) produce biphenyl in excellent yield. The second phase enhances the basicity of the base additive. The enhanced activity in the presence water is particularly surprising.

Substrates are not limited to aromatic halides and boronic acids nor to acrylates. All sp²-substituted reagents which are potential cross coupling partners may be selected for this invention.

WO-A-99/38820 discloses the Heck reaction of an acrylate REM resin (Morphy, J. R.; Rankovic, Z.; Rees, D. C. Tetrahedron Lett. 1996, 37, 3209). Surprisingly such substrates undergo Heck reactions in the presence of palladium (II) acetate and non-fluorinated phosphines of the type defined and exemplified above [preferred examples being selected from tri(t-butyl)phosphine, tri(cyclohexyl)phosphine, tri(o-tolyl)phosphine and 1′-diphenylphosphinobiphenyl] and a base or other reaction-promoting additive (preferred examples being selected from diisopropylethylamine, cesium carbonate, diphylaminomethylpolystyrene, sodium acetate, sodium trifluoroacetate, triethylamine, tri-n-butylamine, perfluorinated trihexylamine, polystyrenemethylammonium carbonate, tetramethylethylenediamine diamine (TMEDA), tetramethyl hexanediamine, and tetraalkylammonium acetates such as tetrabutylammonium acetate. Most preferably the combination palladium (II) acetate, tri(z-butyl)phosphine, iodobenzene, diisopropylethylamine and the acrylate REM resin in compressed carbon dioxide afforded after, cleavage from the resin, cinnamic acid in 98% yield.

Similarly, we also found that Suzuki cross coupling reactions of a selection of halo-vinyl and iodo- and bromo-aryl substituted compounds attached to a Merrifield or Wang resin through an ester linker can be effected with reagents selected from a list of aryl and vinylboronic acids and the above ligand-base-catalyst combinations. Most preferably the combination palladium (II) acetate, tri(t-butyl)phosphine, 4-iodobenzenecarboxylic (ester link to Merrifield resin), 4-methylbenzeneboronic acid and diisopropylethylamine gave 4′-methylbiphenyl-4-carboxylic acid in excess of 80% yield.

Thus, based on these findings, it is a preferred feature of all three aspects of the present invention that the palladium-mediated carbon-carbon bond-forming reactions in compressed carbon dioxide as a solvent are conducted using at least one substrate of the carbon-carbon bond-forming reaction that is bound to a solid polymer support. Examples of suitable polymer supports are the same as those discussed above for the polymer-supported bases and reagents.

Another surprising finding is that when Heck and Suzuki coupling procedures are conducted according to the third aspect of the present invention, they are effective on aryl bromide substrates as well as aryl iodides when the combination tri(t-butyl)phosphine and palladium (U) acetate is used.

We have also found that the palladium-mediated homo-coupling of o-tolylboronic acid can be carried out in compressed carbon dioxide. This is an important and surprising advantage as halide ions are corrosive for the stainless steel vessels necessary for carbon dioxide as solvent. Therefore Heck reactions may be carried out with reactive boronic acids for the initial palladium insertion followed by coupling with an sp²-partner. Initiation of Suzuki reactions with a reactive boronic acid followed by cross coupling with a less reactive boronic acid should also be possible. Thus, in a further aspect of the present invention there is provided a palladium-catalysed Suzuki or Heck reaction in compressed carbon dioxide as a solvent wherein both of the substrates being combined in said reactions are boronic acids. The reagents and reaction conditions may preferably be as defined and exemplified above for the first, second and third aspects of the present invention (e.g. the bases and solubilising ligands can be the solid polymer-supported bases and solubilising ligands defined in the first aspect of the invention, the reactions can be performed in the presence of a tetra alkylammonium acetate as defined in the second aspect of the present invention and the reactions can be carried out using the non-fluorinated phosphine ligands defined in the third aspect of the present invention).

### BRIEF DESCRIPTION OF THE DRAWINGS

The present may be further understood by consideration of the following embodiments of the present invention, with reference to the following drawing in which:

FIG. 1 is a flow diagram of a reactor for a Suzuki reaction carried out under continuous flow conditions in accordance with one aspect of the present invention.

### EXAMPLE 1

### Heck Reactions Using Pd (II) Salts in Combination with Non-Fluorinated Phosphines

Tri-t-butylphosphine (20 mg, 0.1 mmol), palladium (II) acetate, (11 mg, 0.05 mmol), iodobenzene (204 mg, 1 mmol), butyl acrylate (141 mg, 1.1 mmol), and triethylamine (0.121 mg, 1.2 mmol), were placed in a 10 cm³ stainless steel cell under an atmosphere of nitrogen. The cell was sealed, removed from the glove-box and connected to a purged carbon dioxide line. The cell was then charged with carbon dioxide to approximately 800 psi (two thirds full of carbon dioxide.) The suspended reagents were magnetically stirred as the cell was heated to 100° C., 3000 psi. The reagents were stirred at this temperature and pressure for 40 h and the cell was then allowed to cool to room temperature. The contents of the cell were vented into ether (100 cm³) and once atmospheric pressure had been reached the cell was opened and washed out with dichloromethane (20 cm³). The organic fractions were combined and concentrated in vacuo to give the crude product. The product, trans-butyl cinnamate was purified by flash column chromatography on silica gel, eluting with dichloromethane to give an off-white crystalline solid (160 mg, 78%). δH (250 MHz; CDCl₃) 7.68 (1H, d, PhCH═, J 16 Hz), 7.52-7.54 (2H, m, o-Ph), 7.37-7.39 (3H, m, m/p-Ph) 6.44 (1H, d, ═CHCOOBu, J 16 Hz), 4.21 (2H, t, OCH2, J 6.7 Hz), 1.69 (2H, m, OCH₂CH₂), 1.44 (2H, m, OCH₂CH₂CH₂), 0.97 (3H, t, CH₂CH₃ J 7.4 Hz).

Further Heck reactions were conducted in supercritical carbon dioxide following the general process above using various halobenzenes and acrylate esters as the substrates, various bases, palladium (II) acetate (5 mol %) as the catalyst and and tri-t-butylphosphine (10 mol %). The results are as shown in Table 1 below.

TABLE 1
Heck reaction of acrylate esters with halobenzenes
using Pd(II) acetate (5 mol %) and
tri-t-butylphosphine (10 mol %) in sc CO₂

| Entry | Halide | Acrylate | Base (1.2 equiv.) | Time | T/° C. | Yield/% |
|---|---|---|---|---|---|---|
| 1 | Iodo | Methyl | DIPEA | 16 h | 100 | 77 |
| 2 | Iodo | Butyl | DIPEA | 16 h | 100 | 92^{§} |
| 3 | Iodo | Methyl | Cs₂CO₃ | 40 h | 100 | 57 |
| 4 | Iodo | Methyl | NEt₃ | 40 h | 100 | 68 |
| 5 | Iodo | Butyl | NEt₃ | 40 h | 100 | 78 |
| 6 | Bromo | Methyl | DIPEA | 16 h | 100 | 40 |
| 7 | Chloro | Methyl | DIPEA | 16 h | 100 | trace |

^{§}10 mol % catalyst Pd(II) and 20 mol % phosphine; DIPEA is diisopropylethylamine

### EXAMPLE 2

### Heck Reactions of Aryl Halides with Butyl Acrylate in the Presence of Merrifield Phosphines

Diphenylphosphinomethyl polystyrene (obtained from Nova Biochem; 3.30 g, 4.78 mmol 1.45 mmol/g based on manufacturer's loading) and palladium acetate (226 mg, 1 mmol) were stirred in dichloromethane (50 ml) for 24 h. The resulting resin was filtered (sinter), washed [dichloromethane (6×), ethanol (6×) diethyl ether (6×)] and dried to give 3.89 g of resin. Bromobenzene (0.2 ml, 0.19 mmol), butyl acrylate (0.4 ml, 2.6 mmol), tetra n-butylammonium acetate (1 g, 3.4 mmol) and the resin-bound palladium catalyst (100 mg, 30 μmol Pd) were sealed in a stainless steel pressure vessel. The vessel was half-filled with liquid CO₂ (ca. 800 psi) and the mixture was heated at 120° C. (ca. 3000 psi) for 16 h. The cell was cooled and the CO₂ vented into a beaker containing ethyl acetate. The remnants from the cell were washed (ethyl acetate) and pooled with the vented solution. The mixture was filtered and the solvent was removed under reduced pressure to yield a residue (1.713 g). 1,4-Dimethoxybenzene (370 mg) was added as an internal standard and the mixture was dissolved in acetonitrile. The solution was diluted so that the final concentration of standard was less than 1 mg/ml. HPLC determination of the yield of the product n-butyl cinnamate was 100%.

Calibration of the product and internal standard had previously been carried out in a separate experiment. Experiments were carried out a Supelcosil ABZ+ (330×4.6 mm, 3 μm particle size) column: Initial 19:1% water/acetonitrile [symbol] 1:19 water/acetonitrile over 10 min at a rate of 1 ml min⁻¹.

A similar procedure was carried out using different halobenzenes and bases/additives according to Scheme 2 below. The results are shown in Table 2.

Scheme 2 illustrates the use of polymer supported phosphines as exemplified in Table 2.

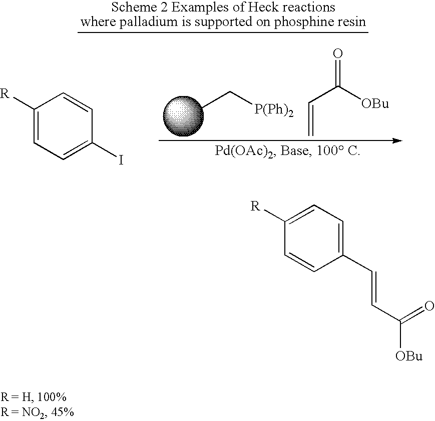

TABLE 2

| | | Pd(OAc)₂ | | Temp | | |
|---|---|---|---|---|---|---|
| Entry | X | mol % | Base or Additive | ° C. | Time h | Yield % |
| 1 | Br | 3 | 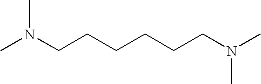 | 120 | 16 | 41^{a} |
| 2 | Cl | 3 | 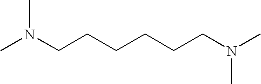 | 120 | 16 | 2^{a} |
| 3 | Br | 3 | 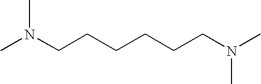 | 100 | 16 | 14^{a} |
| 4 | Br | 3 | 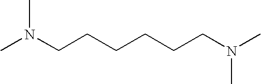 | 80 | 16 | 4^{a} |
| 5 | I | 3 | 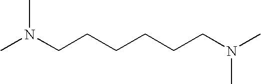 | 80 | 16 | 84 |
| 6 | Br | 3 | (C₄H₉)₄NOAc | 120 | 16 | 100^{a} |
| 7 | Br | 3 | (C₂H₅)₄NOAc.4H₂O | 120 | 16 | >80^{a} |
| 8 | Br | 3 | (C₄H₅)₄NOAc.4H₂O | 100 | 16 | 30^{a} |
| 9 | Br | 3 | (C₄H₉)₄NCl | 100 | 16 | 21^{a} |

^{a}Yield determined by HPLC using internal standard (1,4-dimethoxybenzene)

These results demonstrate clearly that excellent yields can be obtained using polymer-supported phosphine ligands that contain no fluorine. The results obtained when tetraalkylammonium acetates were used were particularly good and much better than those obtained with the corresponding tetra-alkylammonium chloride.

### EXAMPLE 3

### Heck Reactions of Aryl Iodides with Butyl Acrylate Under Phosphorus Free Conditions in the Presence of Polymer-Supported Amines

For cross-coupling reactions to have useful application in the pharmaceutical industry, it is essential that phosphine content be kept to a minimum. We performed a series of Heck reactions using polymer-supported amine bases to see whether it was possible to obtain good yields in the absence of phosphine ligands.

Iodobenzene (0.35 ml, 3.1 mmol), palladium acetate (22 mg, 0.07 mmol), butyl acrylate (0.6 ml, 4 mmol) and diethylaminopolystyrene (1 g, 3.2 mmol of active reagent) were placed in a 10 ml stainless steel pressure reactor. The sealed cell was charged with liquid carbon dioxide (dry and oxygen free) to a pressure of 1000 psi and the cell was heated at 100° C. for 16 h. The reaction was cooled, and the resulting dark green resin was filtered whilst being rinsed with alternate amounts of ethyl acetate and ether. The combined solvents were removed under reduced pressure, and the residue was chromatographed on a flash silica gel column eluted with ethyl acetate-hexane (1:19) to give (E)-butyl cinnamate (580 mg, 95%).

Further Heck reactions were performed under similar conditions according to the general Scheme 3 below. The results of these experiments were highly promising, as shown in Table 3 below.

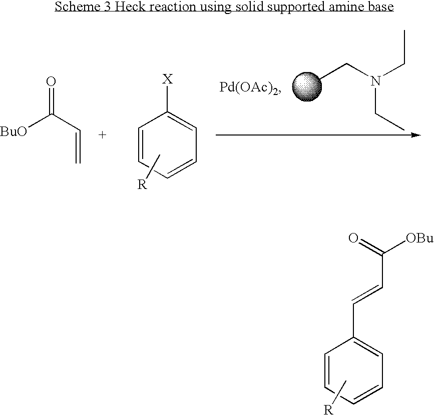

TABLE 3
Solid supported Heck reactions carried
out in the absence of phosphine.

| | | | Approx mol | | Temp | Yield |
|---|---|---|---|---|---|---|
| | X | R | % Pd | Time (h) | (° C.) | (%) |
| 1 | I | 4-H | 5 | 16 | 100 | 95^{a} |
| 2 | I | 4-NO₂ | 5 | 16 | 100 | 92^{a} |
| 3 | I | 4-COMe | 5 | 16 | 100 | 87^{a} |
| 4 | I | 4-OMe | 5 | 72 | 100 | 78^{a} |
| 5 | I | 4-OH | 5 | 16 | 60 | 69^{a} |
| 6 | I | 4-I | 5 | 16 | 100 | 83^{a} |
| 7 | I | 2-OH | 5 | 16 | 100 | 90^{b} |
| 8 | I | 2-CH₂OH | 5 | 16 | 100 | 60 |
| 9 | Br | 4-NO₂ | 5 | 16 | 100 | 93^{a} |

^{a}Yield based on recovery after chromatography;
^{b}Irradiation of the crude reaction product, afforded coumarin in 70% yield.

The use of polymer supported amines facilitated the Heck reaction in supercritical carbon dioxide in the absence of either phosphine or fluorine-substituted solubilising groups. An inherent feature of this invention is that the heterogeneous reaction with palladium catalyst leaves the catalyst embedded in the polystyrene matrix. This invention therefore shows the potential for a continuous flow process using recycled palladium/resin.

### EXAMPLE 4

Suzuki Reactions of Arylboronic Acids with Pd(II) Acetate, a Non-Fluorinated Phosphine and Several Bases

4(a) Biphenyl

A 10 cm³ stainless steel cell was charged with iodobenzene (0.210 g, 1.03 mmol), phenyl boronic acid (0.366 g, 3.0 mmol), palladium (II) acetate, (0.011 g, 0.05 mmol), tri(o-tolyl)phosphine (0.030 g, 0.10 mmol), caesium carbonate (0.978 g, 3.0 mmol) and water (1 cm³). The cell was then connected to the carbon dioxide line and charged with carbon dioxide to approximately 460 psi (half full of carbon dioxide.) The cell was heated to 120° C., and the pressure adjusted to 1650 psi by the addition of more carbon dioxide. The reagents were stirred at this temperature and pressure for 16 h and the cell was then allowed to cool to room temperature. The contents of the cell were vented into ethyl acetate (100 cm³) and once atmospheric pressure had been reached the cell was opened and washed out with further ethyl acetate (50 cm³). The organic fractions were combined and washed with water (30 cm³) then brine (30 cm³) and dried over anhydrous magnesium sulphate. The filtrate was concentrated in vacuo to give the crude product which was purified by flash column chromatography on silica gel, eluting with hexane to give a white crystalline solid (0.143 g, 90%). mp 69-72° C. δH (250 MHz; CDCl₃) 7.60 (4H, d, o-Ph, J 7.8 Hz), 7.45 (4H, dd, m-Ph, J 7.8, 7.2 Hz), 7.35 (2H, t, p-Ph, J 7.2 Hz). δC (400 MHz; CDCl₃) 141.26 (quaternary Ph) 128.74 (m-Ph), 127.25, 127.17 (o/p-Ph).

4(b) 4-Nitrobiphenyl

A 10 cm³ stainless steel cell was charged with 4-bromonitrobenzene (0.202 g, 1.00 mmol), phenyl boronic acid (0.122 g, 1.0 mmol), palladium (II) acetate, (0.002 g, 0.01 mmol), tricyclohexylphosphine (0.006 g, 0.02 mmol) and caesium carbonate (0.326 g, 1.0 mmol). The cell was then connected to the carbon dioxide line and charged with carbon dioxide to approximately 900 psi (half fill of carbon dioxide.) The cell was heated to 110° C., and the pressure adjusted to 3000 psi by the addition of more carbon dioxide. The reagents were stirred at this temperature and pressure for 16 h and the cell was then allowed to cool to room temperature. The contents of the cell were vented into ethyl acetate (100 cm³) and once atmospheric pressure had been reached the cell was opened and washed out with further ethyl acetate (50 cm³). The organic fractions were combined and concentrated in vacuo, then adsorbed onto silica and purified by flash column chromatography on silica gel, eluting with 90:10 hexane:ethyl acetate to give 4-nitrobiphenyl as a white crystalline solid (190 mg, 95%). δH (250 MHz; CDCl₃) 8.31 (2H, d, o-Ar, J 8.9 Hz), 7.74 (2H, d, m-Ar, J 8.9 Hz), 7.63 (2 H, d, o′-Ar), J 6.9), 7.45-7.54 (3H, m, m′-Ar and p′-Ar). δC (250 MHz; CDCl₃) 147.6, 147.1, 138.8, 129.2, 128.9, 127.8, 127.4, 124.1.

The two examples above demonstrate that the Suzuki reaction can be conducted using a triarylphosphine or tricycloalkylphosphine ligand to give a biaryl product such as that shown in Scheme 4 which can be simply isolated by washing and extraction in compressed carbon dioxide, followed by venting the pressure. Further reactions were conducted using according to this general procedure using other halobenzenes, boronic acids, bases and phosphine ligands, including a trialkylphosphine ligand. The results are shown in Table 4 below. This operation shows considerable promise for continuous flow manufacturing in small bore carbon dioxide reactors where the catalyst and amine salt remain behind in the reactor vessel and the product is extracted by carbon dioxide. Tis process should be equally possible using the tetralkylammonium additive technique.

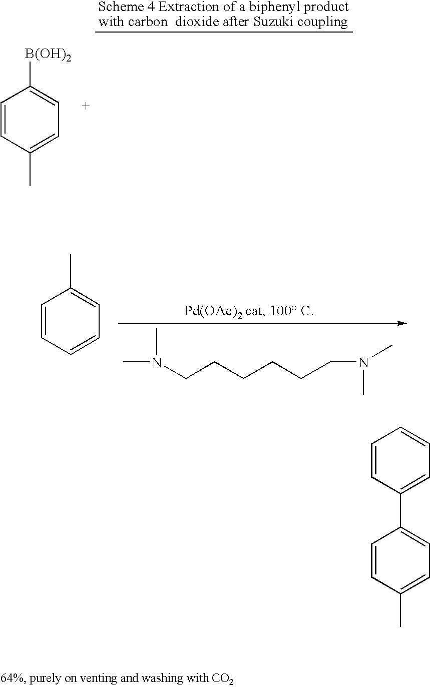

TABLE 4
Suzuki reaction of aryl halides with aryl boronic acids in the presence of Pd(II)
acetate (5 mol %) and various phosphines (10 mol %.) and bases (2 equiv.)

| | | Boronic | | | Time/ | T/ | Yield/ |
|---|---|---|---|---|---|---|---|
| Entry | Halide | acid | Phosphine | Base | h | ° C. | % |
| 1 | Iodo | Phenyl | P(t-Bu)₃ | DIPEA | 16 | 100 | 68 |
| 2 | Iodo | Tolyl | P(t-Bu)₃ | DIPEA | 16 | 100 | 73 |
| 3 | Iodo | Tolyl | P(t-Bu)₃ | 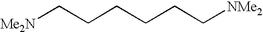 | 16 | 120 | 76 |
| 4 | Bromo | Tolyl | P(t-Bu)₃ | DIPEA | 16 | 100 | 54 |
| 5 | Bromo | Tolyl | P(t-Bu)₃ | Cs₂CO₃ | 16 | 100 | 52 |
| 6 | Chloro | Tolyl | P(t-Bu)₃ | DIPEA | 64 | 100 | 19 |
| 7 | Iodo | Phenyl | P(o-tolyl)₃ | Cs₂CO₃/H₂O | 16 | 120 | 90 |
| 8 | Bromo | Phenyl | P(o-tolyl)₃ | Cs₂CO₃/H₂O | 64 | 110 | 96 |
| 9 | Bromo | Tolyl | P(o-tolyl)₃ | Cs₂CO₃/H₂O | 16 | 110 | 73 |
| 10 | Bromo | Phenyl | P(cy)₃ | Cs₂CO₃/H₂O | 16 | 110 | 73 |
| 11 | Bromo | Phenyl | P(cy)₃ | Cs₂CO₃/H₂O | 40 | 110 | 90 |
| 12 | Bromo | Phenyl | P(cy)₃ | NBu₄OAc | 16 | 110 | 72 |
| 13 | Bromo | Tolyl | P(cy)₃ | NBu₄OAc | 64 | 110 | 84 |

Buchwald (Old., D. W.; Buchwald, S. J. Am. Chem. Soc. 1998, 120, 9722) and Fu (Littke, A. F.; Fu, G. C. Angew. Chem. Int. Ed. 1998, 37,3387) have exploited tri-t-butylphosphine in a variety of cross-coupling reactions mediated by Pd(0). In this invention it has surprisingly been found that not only tri-t-butylphosphine, but also tricyclohexylphosphine and tri-o-tolylphosphine can be used for the successive promotion of Suzuki coupling reactions with Pd(OAc)₂ in supercritical carbon dioxide with aryl iodides, bromides, and chlorides, as is demonstrated in Table 4 above.

### EXAMPLE 5

### Heck Reactions on a Substrate Supported on Solid Phase

WO-A-99/38820 discloses the Heck reaction of an acrylate attached to a Merrifield resin on which solubilising fluorinated phosphines were used to solubilise the palladium catalyst. This research has the potential to exploit the swellability of such resins and to carry out rapid parallel synthesis and combinatorial chemistry with solid phase supported substrates in supercritical carbon dioxide. Commercially available REM resin was treated with a variety of aromatic aryl iodides, Pd(II) acetate, and an ante base at either 40 or 80° C. These results demonstrate the surprisingly efficient heterogeneous Heck coupling reaction with tri-t-butylphosphine as the ligand. The following gives a number of fall worked example of the conditions used for the preparation of various cinnamic acids. A similar approach was then used for other palladium sources, bases, temperatures and reaction times as shown in Table 5.

(a) trans-Cinnamic Acid

Commercially available REM resin (500 ma, 0.44 mmol; obtained from Nova Biochem), iodobenzene (102 mg, 0.5 mmol), palladium trifluoroacetate (17 mg, 0.05 mmol), diisopropylethylamine (0.1 ml, 0.55 mmol) and t-tributylphospine (28 mg, 0.14 mmol) were placed in a stainless steel cell and suspended in supercritical CO₂ at 40° C. for 40 h. After this time, the cell was cooled and the product filtered, whilst washing with CH₂Cl₂ (6×), water (6×), EtOAc (3×) and Et₂O (3×)—to give the modified resin (632 mg). The resin (620 mg) was stirred in a KOH solution [3 ml, 1.5M solution in TFA/MeOH/H₂O (2:2:1)] for 20 h. This was filtered and the solvent removed under reduced pressure. The residue was redissolved in ethyl acetate and was washed with dil. HCl (1M, water and brine. This was dried (MgSO₄) and the solvent removed under reduced pressure. The residue was chromatographed (silica gel, ethyl acetate—toluene (2:8) as the eluent) to give trans-cinnamic acid (58 mg, 93%). Solid phase IR of the remaining resin indicated complete saponification of the ester linker.

(b) Trans-Cinnamic Acid

The REM resin (500 mg, 0.44 mmol 100-200 mesh), iodobenzene (0.12 ml, 1.2 mmol), Pd(OAc)₂ (10 mg, 0.05 mmol), tri-1-butylphosphine (60 mg, 0.3 mmol) and diisopropylethylamine (0.08 ml, 0.46 mmol) were sealed under an atmosphere of carbon dioxide (ca. 800 psi). The reaction was heated at 80° C. for 16 h. The reaction was cooled and resin filtered through a sinter whilst sequentially washing with dichloromethane (4×), H₂O (4×), EtOAc (6×) and diethyl ether (6×). The resin was stirred in a solution of KOH (10 ml, 1.5 M solution in THF/H₂O/MeOH) for 16 h. The solution was acidified with dil. HCl (1 M) and the product extracted with EtOAc. The organic phase was washed (water, brine), dried (MgSO₄) and the reduced product chromatographed (silica gel, ethyl acetate-toluene (1:3)) to give cinnamic acid (66 mg, 98%).

(c) 4-Nitrocinnamic Acid

The REM resin (500 mg, 0.44 mmol), 4-nitroiodobenzene (125 mg, 0.5 mmol), Pd(OAc)₂ (11 mg, 0.05 mmol), tri-t-butylphosphine (75 ml, 0.37 mmol) and NEt₃ (0.3 ml, 2.8 mmol) were sealed under an atmosphere of carbon dioxide (ca. 800 psi). The reaction was heated at 80° C. for 16 h. The reaction was cooled and the resin filtered through a sinter whilst sequentially washing with dichloromethane (4×), H₂O (4×), EtOAc (6×) and diethyl ether (6×).

The resin was stirred in a solution of KOH (10 nm 1.5 M solution in THF/H₂O/MeOH) for 16 h. The solution was acidified with dil. HCl (1 M) and the product extracted with EtOAc. The organic phase was washed (water, brine), dried (MgSO₄) and the solvent removed under reduced pressure to give the crude nitro product (90 mg, ca 100%).

(d) 4-Hydroxycinnamic Acid and 4-Acetylcinnamic Acid

The REM resin (1 g, 0.88 mmol), 4-iodophenol (110 mg, 0.44 mmol), 4-iodoacetophenone (123 mg, 0.44 mmol), Pd(OAc)₂ (11 mg, 0.05 mmol), tri-t-butylphosphine (25 ml, 13 mmol) and NEt₃ (0.28 ml, 2.2 mmol) were sealed under an atmosphere of carbon dioxide (ca. 800 psi). The reaction was heated at 80° C. for 16 h (Ca 1700 psi). The reaction was cooled and the resin filtered through a sinter whilst sequentially washing with dichloromethane (4×), H₂O (4×), EtOAc (6×) and diethyl ether (6×). The resin was dried (1.21 g).

The resin was stirred in a solution of KOH (10 ml, 1.5 M solution in THF/H₂O/MeOH) for 16 h. The solution was acidified with dil. HCl (1 M) and the product extracted with EtOAc. The organic phase was washed (water, brine), dried (MgSO₄) and the solvent removed under reduced pressure. The residue was chromatographed (silica gel, methanol-dichloromethane 1:19 as the eluent) to give 4-hydroxycinnamic acid (42 mg, 58%) followed the acetophenone derivative (ca. 50 mg, 63%).

Using the approach set out in the above examples, the results for other palladium sources, bases, temperatures and reaction times are as shown in Table 5.

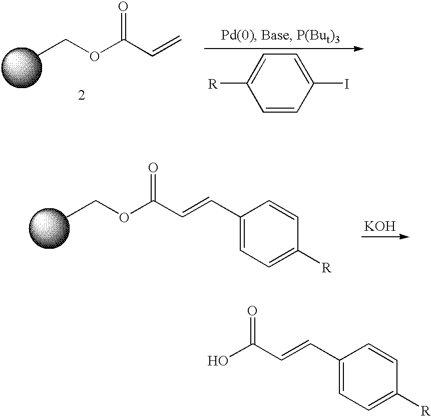

TABLE 5
Investigations into solid supported reactants in scCO₂

| | | | | Time | | |
|---|---|---|---|---|---|---|
| | R | Pd Source | Base | (h) | Temp (° C.) | Yield (%) |
| 1 | H | Pd(OCOCF₃)₂ | NEt₃ | 18 | 40 | 60^{a} |
| 2 | H | Pd(OCOCF₃)₂ | NEt₃ | 40 | 40 | 80^{a} |
| 3 | H | Pd(OCOCF₃)₂ | DIPEA | 40 | 40 | 92^{a} |
| 4 | H | Pd(OCOCF₃)₂ | NEt3 | 40 | 80 | 93^{a} |
| 5 | H | Pd(OCOCF₃)₂ | DIPEA | 40 | 80 | 95^{a} |
| 6 | H | Pd(OAc)₂ | DIPEA | 16 | 80 | 98^{a} |
| 7 | NO₂ | Pd(OAc)₂ | NEt₃ | 16 | 80 | 42^{b} |
| 8 | COMe | Pd(OAc)₂ | NEt₃ | 16 | 80 | 70^{b} |

^{a}Yield determined by cleavage from resin after purification by chromatography;
^{b}Yield from crude product NMR;
^{c}All experiments were carried out with 10 mol % Pd

Scheme 5 Heck reactions carried out on commercially available REM resin

Several aspects are noteworthy from the results in Table 5. These reactions are the first reactions of their kind to be carried out in supercritical CO₂. Changing the source of palladium did not influence the yields to any great extent, however; it was noted that DIPEA (diisopropylethylamine) was a more effective base than triethylamine. The reactions are feasible at mild temperatures.

### EXAMPLE 6

### Suzuki Reactions on a Substrate Supported on Solid Phase

(a) Preparation of the Resins

(i) Merrifield resin [5 g, 6.2 mmol, 1.24 mmol/g based on manufacturers specification (Nova Biochem)], p-bromobenzoic acid (2 g, 10 mmol), Cs₂CO₃ (1.51 g, 4.6 mmol) and KI (0.5 g, 3.0 mmol) in dimethylformamide (80 ml) were heated at 80° C. for 16 h. The cooled solution was filtered through a sinter whilst washing sequentially with EtOAc (3×), H₂O (3×), EtOAc (3×), dichloromethane (3×), ethanol (6×) and diethyl ether (6×). The residue was dried (5.8 g), δₘₐₓ/cm⁻¹ (FTIR solid phase) 1719 (CO).

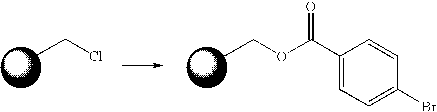

(ii) The identical reaction was carried out using p-iodobenzoic acid giving similar results.

(iii) Wang Resin [3.09 g, 4 mmol, 1.3 mmol/g based on manufacturers specification (Nova Biochem)], p-bromobenzoic acid (1.6 g, 8 mmol), diisopropyldiimide (1 ml, 6.4 mmol) and dimethylaminopyridine (DMAP) (48 mg, mmol) in dichloromethane (50 ml) were stirred for 20 h. The residue was filtered through a sinter whilst washing sequentially with dichloromethane (6×), H₂O (4×), dichloromethane (3×), EtOAc (6×), ethanol (6×) and diethyl ether (6×). The residue was dried (3.44 g); δₘₐₓ/cm⁻¹ (FTIR solid phase) 1720 (CO). 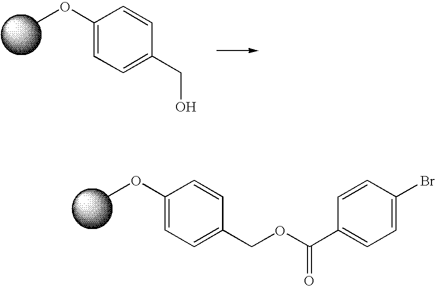
(b) Use of the Prepared Resins in Suzuki Reactions
(i) 4 Phenylbenzoic Acid

The Merrifield resin prepared in (a)(i) above (500 mg, ca. 0.65 mmol), phenyl boronic acid (244 mg, 2.0 mmol), diisopropylethylamine (0.19 ml, 1.1 mmol), Pd(TFA)₂ (33 mg, 0.1 mmol) and t-tributylphosphine (50 mg, 0.26 mmol) were placed in a reaction cell and the cell sealed under an atmosphere of carbon dioxide (ca. 800 psi). The reagents were heated at 80° C. for 40 h when the mixture was cooled. The resin was filtered whilst washing sequentially with CH₂Cl₂ (6×), H₂O (6×), EtOAc (3×) and Et₂O (3×). The resin was dried to constant mass (460 mg); δₘₐₓ/cm⁻¹ (FTIR solid phase) 1714 (CO). The resin thus obtained (440 mg) was stirred in TFA/CH₁₂Cl₂ (1:1, 5 ml) for 6 h. This was filtered whilst washing with CH₂Cl₂. The solvent was removed under reduced pressure and the residue was chromatographed (silica gel, EtOAc/Toluene 3:7) to yield the biaryl product (80 mg, 68% over 3 steps)

(ii) 4-Tolylbenzoic Acid

The Merrifield resin prepared in (a)(i) above (1.8 g, ca. 2.2 mmol), 4-tolyl boronic acid (500 mg, 3.6 mmol), diisopropylethylamine (0.2 ml, 1.1 mmol), Pd(OAc)₂ (10 mg, 0.04 mmol) and t-tributylphosphine (100 mg, 0.5 mmol) were placed in a reaction cell and the cell sealed under an atmosphere of carbon dioxide (ca. 800 psi). The reagents were heated at 80° C. for 16 h when the mixture was cooled. The resin was filtered whilst washing sequentially with CH₂Cl₂ (6×), H₂O (6×), EtOAc (3×) and Et₂O (3×). The resin thus obtained was stirred in a solution of KOH (10 ml, 1.5 M solution in THF/H₂O/MeOH) for 16 h. The solution was acidified with dil. HCl (1 M and the product extracted with EtOAc. The organic phase was washed (water, brine), dried (MgSO₄) and the solvent removed under reduced pressure to give a mixture of starting material and biaryl product (340 mg, 40% by NMR, >85% based on equivalents of diisopropylethylamine).

The results for reaction of freshly prepared iodobenzoate-modified Merrifield resin with commercially available boronic acids gave promising results (Table 6)

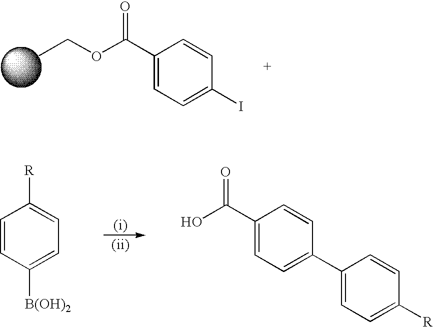

Scheme 6 Reagents and conditions: (i) Pd(OAc)₂, P(t-Bu)₃, base, heat; (ii) KOH, MeOH, THF

TABLE 6
Pilot Suzuki reactions of supported reactants carried out in scCO₂

| | | | Pd | | Time | Temp | |
|---|---|---|---|---|---|---|---|
| Entry | R | Pd Source | (mol %) | Base | (h) | (° C.) | Yield (%) |
| 1 | H | Pd(OCOCF₃)₂ | 10 | DIPEA | 40 | 80 | >65^{a} |
| 2 | Me | Pd(OAc)₂ | 5 | DIPEA | 16 | 80 | >80^{a} |

^{a}Results are based on manufacturer's loading

The aforementioned reactions clearly demonstrate the successful application of heterogeneous cross-coupling reactions of solid supported substrates in compressed CO₂.

### EXAMPLE 7

Continuous Flow Suzuki Reaction in Supercritical CO₂ of an Aryl Halide with Phenylboronic Acid

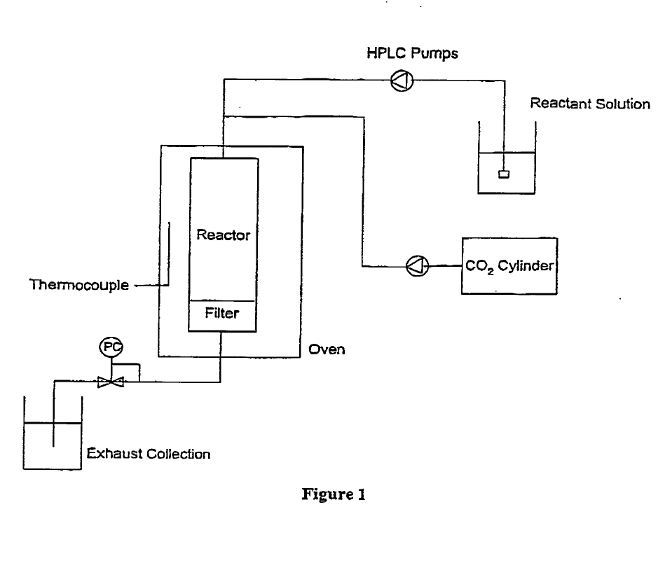

A 50 cm³ stainless steel pressure reactor was fitted with a filter, and connected to three stainless steel injection lines which were pressurised by HPLC pumps (see FIG. 1). An outlet (exhaust) line was connected via a back pressure regulator. The vessel was placed in an oven and was heated to 110° C. CO₂ was charged at a rate of 5 cm³/min until a pressure of 140 kg/cm² (137 bar) was reached. Once the set temperature and pressure were attained, methanol (5 cm³) was added at a rate of 0.5 cm³/min over 10 minutes. Palladium acetate (0.011 g, 0.05 mmol) and tricyclohexylphosphine (0.028 g, 0.10 mmol) were made up in a solution of methanol (5 cm³). This solution was added at a rate of 0.5 cm³/min over 10 minutes, then further methanol (5 cm³) was added at the same rate for a further 10 minutes. A solution of bromobenzene (0:162 g, 1.03 mmol), phenylboronic acid (0.122 g, 1.00 mmol) and tetrabutylammonium acetate (0.301 g, 1.00 mmol) in methanol (20 cm³) was prepared. The rate of CO₂ addition was adjusted to 2 cm³/min and the reagent solution was added at a rate of 0.1 cm³/min. Once addition was complete methanol (5 cm³) was added at the same rate of 0.1 cm³/min to flush the HLC line. Once this addition was complete the reactor was depressuzised. All exhaust from the vessel was vented through ethyl acetate (150 cm³), which was collected, reduced in volume in vacuo and subjected to column chromatography on silica gel eluting with 100% isohexane to give the product, biphenyl (0.078 g, 51%) as a white crystalline solid.

Further reagents were reacted under continuous flow conditions. The results are shown in Table 7 below. The surprising success of the Suzuki coupling of aryl bromides under conditions where the reagents are in such short contact times with the catalyst is noteworthy.

TABLE 7
Continuous flow Suzuki reaction in scCO₂ of
an aryl halide with phenylboronic acid using
NBu₄OAc and a catalyst comprising Pd(OAc)₂/PCy₃

| Entry | Aryl bromide | Yield (%) |
|---|---|---|
| 1 | bromobenzene | 51 |
| 2 | 4-bromofluorobenzene | 65 |
| 3 | 4-bromoanisole | 26 |

### EXAMPLE 8

### Homocoupling of Phenylboronic Acid to Give Biphenyl

Phenylboronic acid (0.488 g, 4 mmol), tetrabutylammonium acetate (1.204 g, 4 mmol) and palladium(II) acetate (0.022 g, 0.01 mmol) were added to a stainless steel pressure reactor vessel. The vessel was charged with carbon dioxide and was then pressurised to 3000 psi at a temperature of 110° C. The reaction was allowed to proceed under these conditions for 16 h. The vessel was cooled to 25° C., and the contents were vented into ethyl acetate (100 cm⁻³). The cell was rinsed out with further ethyl acetate (50 cm³). Evaporation of the combined organic solvent and recrystallisation of the crude product from hexane afforded biphenyl (0.028 g, 9%).

1. A palladium-catalysed carbon-carbon bond forming reaction in compressed carbon dioxide as a solvent wherein at least one of the reagents used in said reaction is bound to a solid polymer support.

2. A palladium-catalysed carbon-carbon bond forming reaction in compressed carbon dioxide as a solvent according to claim 1 wherein said reaction is selected from the group consisting of Heck, Suzuki, Sonogashira and Stille reactions.

3. A palladium-catalysed carbon-carbon bond forming reaction in compressed carbon dioxide as a solvent according to claim 1 or claim 2 wherein said polymer-bound reagents are selected from polymer-supported bases and polymer-supported solubilising ligands.

4. A palladium-catalysed carbon-carbon bond forming reaction in compressed carbon dioxide as a solvent according to claim 3 wherein said polymer-supported base is a polymer-supported amine base.

5. A palladium-catalysed carbon-carbon bond forming reaction in compressed carbon dioxide as a solvent according to claim 4 wherein said polymer-supported amine base is a polymer having supported monoalkylaminoalkyl groups or a polymer having supported dialkylaminoalkyl groups wherein each alkyl group is the same or different and has from 1 to 6 carbon atoms.

6. A palladium-catalysed carbon-carbon bond forming reaction in compressed carbon dioxide as a solvent according to any one of claims 3 to 5 wherein said supporting solid polymer is selected from polystyrenes and macroreticular resins.

7. A palladium-catalysed carbon-carbon bond forming reaction in compressed carbon dioxide as a solvent according to any one of claims 3 to 6 wherein said polymer-supported base is selected from dialkylaminoalkylpolystyrenes and dialkylamino-macroreticular resins.

8. A palladium-catalysed carbon-carbon bond forming reaction in compressed cart dioxide as a solvent according to claim 7 wherein said polymer-supported base is selected from diethylaminomethylpolystyrene, diethylaminomethyl-macroreticular resin and disopropylmethylaminopolystyrene.

9. A palladium-catalysed carbon-carbon bond forming reaction in compressed carbon dioxide as a solvent according to claim 7 wherein said polymer-supported base is diethylaminomethylpolystyrene.

10. A palladium-catalysed carbon-carbon bond forming reaction in compressed carbon dioxide as a solvent according to any one of claims 1 to 9 wherein said polymer-supported solubilising ligand is selected from polymer-supported phosphine ligands.

11. A palladium-catalysed carbon-carbon bond forming reaction in compressed carbon dioxide as a solvent according to claim 10 wherein said supporting polymer is selected from polystyrenes and macroreticular resins.

12. A palladium-catalysed carbon-carbon bond forming reaction in compressed carbon dioxide as a solvent according to any one of claims 1 to 11 wherein said phosphine ligands on said solid polymer supports are selected from phosphine ligands that have at least one fluoro-substituted aliphatic or aromatic substituent, and phosphine ligands that do not have any fluorinated substituents but do have at least one substituent selected from alkyl groups, cycloalkyl groups and aryl groups.

13. A palladium-catalysed carbon-carbon bond forming reaction in compressed carbon dioxide as a solvent according to claim 12 wherein said phosphine ligands on said solid polymer supports are selected from diarylphosphinoalkyl groups, dialkylphospinoalkyl groups and dicycloalkylphospinoalkyl groups wherein each alkyl moiety has from 1 to 6 carbon atoms, each cycloalkyl group has from 3 to 8 carbon atoms and each aryl group is a phenyl group that may optionally be substituted with at least one alkyl group having from 1 to 6 carbon atoms.

14. A palladium-catalysed carbon-carbon bond forming reaction in compressed carbon dioxide as a solvent according to claim 10 wherein said polymer-supported phosphine is selected from polystyrenes and macroreticular resins having supported diphenylphosphinoalkyl groups wherein said alkyl groups have from 1 to 6 carbon atoms.

15. A palladium-catalysed carbon-carbon bond forming reaction in compressed carbon dioxide as a solvent according to claim 14 wherein said polymer-supported phosphine is diphenylphospinomethylpolystyrene.

16. A palladium-catalysed carbon-carbon bond forming reaction in compressed carbon dioxide as a solvent according to any one of claims 1 to 15 wherein at least one of the substrates of the carbon-carbon bond forming reaction is bound to a solid polymer support.

17. A palladium-catalysed carbon-carbon bond forming reaction in compressed carbon dioxide as a solvent according to any one of claims 1 to 16 wherein said reaction is conducted as a continuous flow reaction.

18. A palladium-catalysed carbon-carbon bond forming reaction in compressed carbon dioxide as a solvent wherein said reaction is performed in the presence of a tetra-alkylammonium acetate wherein each alkyl group is the same or different and has from 1 to 6 carbon atoms.

19. A palladium-catalysed carbon-carbon bond forming reaction in compressed carbon dioxide as a solvent according to claim 18 wherein each all group of said tetra-alkylammonium acetate is the same or different and has from 1 to 4 carbon atoms.

20. A palladium-catalysed carbon-carbon bond forming reaction in compressed carbon dioxide as a solvent according to claim 18 wherein said tetra-alkylammonium acetate is selected from tetraethylammonium acetate and tetra(n-butyl)ammonium acetate.

21. A palladium-catalysed carbon-carbon bond forming reaction in compressed carbon dioxide as a solvent according to claim 18 wherein said tetra-alkylammonium acetate is tetra(n-butyl)ammonium acetate.

22. A palladium-catalysed carbon-carbon bond forming reaction in compressed carbon dioxide as a solvent according to any one of claims 18 to 21 wherein said reaction is selected from the group consisting of Heck, Suzuki, Sonogashira and Stille reactions.

23. A palladium-catalysed carbon-carbon bond forming reaction in compressed carbon dioxide as a solvent according to any one of claims 18 to 22 wherein said reaction is conducted using a solubilising ligand selected from phosphine ligands that have at least one fluoro-substituted aliphatic or aromatic substituent, and phosphine ligands that do not have any fluorinated substituents but, do have at least one substituent selected from alkyl groups, cycloalkyl groups and aryl groups.

24. A palladium-catalysed carbon-carbon bond forming reaction in compressed carbon dioxide as a solvent according to claim 23 wherein said phosphine ligands are selected from triarylphosphinoalkyl groups, trialkylphospinoalkyl groups and tricycloalkylpbospinoalkyl groups wherein each alkyl moiety has from 1 to 6 carbon atoms, each cycloalkyl group has from 3 to 8 carbon atoms and each aryl group is a phenyl group that may optionally be substituted with at least one alkyl group having from 1 to 6 carbon atoms.

25. A palladium-catalysed carbon-carbon bond forming reaction in compressed carbon dioxide as a solvent according to claim 23 wherein said phosphine ligands are selected from tri(t-butyl)phosphine, tri(cyclohexyl)phosphine, tri(o-tolyl)phosphine and 1′-diphenyl-phosphinobiphenyl.

26. A palladium-catalysed carbon-carbon bond forming reaction in compressed carbon dioxide as a solvent according to any one of claims 18 to 22 wherein said reaction is conducted using a polymer-supported solubilising ligand as defined in any one of claims 10 to 15.

27. A palladium-catalysed carbon-carbon bond forming reaction in compressed carbon dioxide as a solvent according to any one of claims 18 to 22 wherein at least one of the substrates of the carbon-carbon bond forming reaction is bound to a solid polymer support.

28. A palladium-catalysed carbon-carbon bond forming reaction in compressed carbon dioxide as a solvent according to any one of claims 18 to 27 wherein said reaction is conducted as a continuous flow reaction.

29. A palladium-catalysed carbon-carbon bond forming reaction in compressed carbon dioxide as a solvent wherein said palladium catalyst does not have any fluorinated phosphine ligands but does have at least one phosphine ligand that has at least one substituent that is selected from the group consisting of tert-alkyl groups having from 4 to 10 carbon atoms, cycloalkyl groups having from 3 to 8 carbon atoms and phenyl groups which can be substituted with at least one alkyl group having from 1 to 6 carbon atoms or 1′-diphenyl-phosphinobiphenyl.

30. A palladium-catalysed carbon-carbon bond forming reaction in compressed carbon dioxide as a solvent according to claim 29 wherein said tert-alkyl substituents for said phosphine ligands are tert-butyl groups, said cycloalkyl substituents for said phosphine ligands are cyclohexyl groups and said optionally substituted phenyl groups for said phosphine ligands are o-tolyl groups.

31. A palladium-catalysed carbon-carbon bond forming reaction in compressed carbon dioxide as a solvent according to claim 29 wherein said phosphines are selected from tri(t-butyl)phosphine, tri(cyclohexyl)phosphine, tri(o-tolyl)phosphine and 1′-diphenylphosphinobiphenyl.

32. A palladium-catalysed carbon-carbon bond forming reaction in compressed carbon dioxide as a solvent according to claim 29 wherein said phosphine is tri(z-butyl)phosphine.

33. A palladium-catalysed carbon-carbon bond forming reaction in compressed carbon dioxide as a solvent according to any one of claims 29 to 32 wherein said reaction is selected from the group consisting of Heck, Suzuki, Sonogashira and Stille reactions.

34. A palladium-catalysed carbon-carbon bond forming reaction in compressed carbon dioxide as a solvent according to any one of claims 29 to 33 wherein said reaction is performed in the presence of a base or other reaction-promoting additive selected from diisopropylethylamine, cesium carbonate, diethylaminomethylpolystyrene, sodium acetate, sodium trifluoroacetate, triethylamine, tri-n-butylamine, perfluorinated trihexylamine, polystrenemethylammonium carbonate, tetramethylethylenediamine diamine (TMEDA), tetramethyl hexanediamine, and tetra-alkylammonium acetates wherein each alkyl group has from 1 to 6 carbon atoms.

35. A palladium-catalysed carbon-carbon bond forming reaction in compressed carbon dioxide as a solvent according to claim 34 wherein said reaction is performed in the presence of a base selected from tetramethyl hexanediamine and cesium carbonate.

36. A palladium-catalysed carbon-carbon bond forming reaction in compressed carbon dioxide as a solvent according to claim 34 wherein said reaction is performed in the presence of a reaction-promoting additive selected from tetra-alkylammonium acetates wherein each alkyl group has from 1 to 6 carbon atoms.

37. A palladium-catalysed carbon-carbon bond forming reaction in compressed carbon dioxide as a solvent according to any one of claims 29 to 36 wherein at least one of the substrates of the carbon-carbon bond forming reaction is bound to a solid polymer support.

38. A palladium-catalysed carbon-carbon bond forming reaction in compressed carbon dioxide as a solvent according to any one of claims 29 to 37 wherein said reactions is conducted as a continuous flow reaction.

39. A palladium-catalysed Suzuki or Heck reaction in compressed carbon dioxide as a solvent wherein both of the substrates being combined in said reactions are boronic acids.
